(19) United States
(12) Patent Application Publication
(10) Pub. No.: US 2026/0306870 A1
(43) Pub. Date: Oct. 8, 2026

(54) HEADWEAR DEVICE FOR PROVIDING LIGHT THERAPY

(71) Applicant: Shenzhen Rainbow Light Medical Equipment Co., Ltd., Shenzhen (CN)

(72) Inventors: Alain Dijkstra, Amstelveen (NL); Cao Jiaming, Shenzhen (CN); Chu Zhanghui, Shenzhen (CN); Wang Meng, Shenzhen (CN); Yin Zhichao, Shenzhen (CN)

(73) Assignee: Shenzhen Rainbow Light Medical Equipment Co., Ltd., Shenzhen (CN)

(21) Appl. No.: 19/632,524

(22) Filed: Mar. 30, 2026

(30) Foreign Application Priority Data
Apr. 2, 2025 (CN) 202520621301X

(51) Int. Cl. A61N 5/06; A61N 5/00

(52) U.S. Cl. CPC A61N 5/0617; A61N 2005/007; A61N 2005/0627; A61N 2005/0647; A61N 2005/0652

(57) ABSTRACT

A light therapy headwear device is disclosed, comprising a headwear device body having an accommodating space for a head of a user, a head circumference adjustment assembly coupled to the headwear device body, and a first light source assembly coupled to the head circumference adjustment assembly for performing light therapy on the back of the head. The head circumference adjustment assembly comprises a regulator and telescoping connecting arms that selectively move the first light source assembly toward or away from the back of the head, thereby regulating thermal energy transfer and light therapy intensity from the first light source assembly to the back of the head. This configuration addresses challenges in existing light therapy headwear devices where heat conduction is difficult to adjust. The headwear device may include additional light sources for the forehead and overhead and upper rear regions, as well as light-transmitting shells for protecting the user.

### TECHNICAL FIELD

The present invention relates to the field of light therapy equipment, and more particularly to a headwear device configured to provide light therapy treatment to the human head.

### BACKGROUND OF THE INVENTION

Light therapy is a non-invasive therapeutic modality that uses specific wavelengths of light to irradiate the human body. Light therapy has been shown to exert beneficial biological effects, including antibacterial and anti-inflammatory action, acceleration of blood circulation, promotion of tissue regeneration, improvement of cellular metabolism, and adjuvant treatment of various diseases and conditions.

When light therapy is to be applied to specific regions of the human body, it is necessary to employ light therapy equipment that is specifically designed and configured for those body regions. The light therapy headwear device is one category of light therapy equipment designed for the head. By irradiating the scalp with therapeutic light, a light therapy headwear device can promote hair growth, stimulate hair follicles, improve scalp circulation, and address conditions such as alopecia and related scalp disorders. Various light therapy headwear devices for scalp treatment are known in the art.

In existing light therapy headwear devices, the light source is typically fixedly mounted to the interior surface of the light therapy headwear device body at a fixed distance from the scalp. The user wears the light therapy headwear device, and the fixed light source irradiates the scalp at this fixed distance. While such an arrangement is mechanically simple, it presents significant limitations, such as the user cannot adjust the distance between the light source and the scalp after the light therapy headwear device has been put on. Because all light sources generate some amount of heat in addition to therapeutic light, the inability to adjust the distance between the light source and the scalp means that the user cannot control the amount of heat transmitted from the light source to the scalp, nor can the user adjust the intensity of the light therapy delivered to the scalp. While some prior art devices address heat through ventilation holes, spacers, or cooling mechanisms, these approaches do not give the user direct control over the light-to-scalp distance. Furthermore, existing head circumference adjustment mechanisms are designed solely for fit and do not simultaneously adjust the distance between a light source and the scalp.

Therefore, there is a need for a light therapy headwear device that allows the user to adjust both the heat and the light therapy intensity transmitted by the light source to the scalp, offering enhanced safety, comfort, and therapeutic efficacy compared to prior art light therapy headwear devices. The present invention provides a light therapy headwear device that overcomes the limitations of the conventional devices. The light therapy headwear device allows the user to adjust the distance between the first light source assembly and the back of their head.

### OBJECTS OF THE INVENTION

Some objects of the invention are as follows:

An object of the present invention is to provide a light therapy headwear device that enables the user to selectively adjust the spacing distance between a first light source assembly and the back of the head, thereby regulating thermal energy transfer and light therapy intensity from the first light source assembly to the scalp.

Another object of the present invention is to provide a light therapy headwear device with a head circumference adjustment assembly that can be adjusted to accommodate users having different head circumferences, so that the light therapy headwear device can be stably and comfortably worn on heads of various sizes.

Another object of the present invention is to provide a light therapy headwear device having a first light source assembly for treating the back of the head, a second light source assembly for treating the forehead region of the head, and a third light source assembly for treating the overhead and upper rear regions of the head.

Another object of the present invention is to provide a light therapy headwear device wherein each light source assembly is separated from the user's scalp by a light-transmitting shell, which transmits therapeutic light to the scalp while reducing the direct conduction of heat from the light source to the head.

Another object of the present invention is to provide a light therapy headwear device with a support assembly comprising a plurality of support columns that form ventilation gaps between the light-transmitting shells and the user's scalp, facilitating cooling airflow within the accommodating space.

Another object of the present invention is to provide a light therapy headwear device wherein the support columns are configured to contact the scalp of the user during use, providing a gentle massaging effect and enhancing wearing comfort.

Another object of the present invention is to provide a light therapy headwear device with an integrated power supply and controller, wherein the controller includes a power indicator light and a buzzer, to allow the user to conveniently manage light therapy sessions and to prevent excessive or prolonged exposure.

Another object of the present invention is to provide a light therapy headwear device with a buffer component attached to the interior of the headwear device body via a hook-and-loop fastener, providing cushioning and comfort protection for the user's forehead during use.

Yet another object of the present invention is to provide a light therapy headwear device that is safe, user-friendly, durable, and suitable for home use.

### SUMMARY OF THE INVENTION

According to a first aspect of the invention, a light therapy headwear device is provided. The light therapy headwear device comprising: a headwear device body having an accommodating space for a head of a user and an accommodation opening at one end of the headwear device body along a first direction; a head circumference adjustment assembly coupled to the headwear device body proximate to the accommodation opening; and a first light source assembly coupled to the head circumference adjustment assembly and configured to perform light therapy on a back of the head of the user, wherein the head circumference adjustment assembly is mechanically coupled to the first light source assembly and configured such that adjustment of the head circumference adjustment assembly causes displacement of the first light source assembly relative to the back of the head of the user to selectively adjust a spacing distance between the first light source assembly and the back of the head of the user, thereby regulating thermal energy transfer and light therapy intensity from the first light source assembly to the back of the head of the user.

In one embodiment of the invention, the head circumference adjustment assembly comprises a regulator and two connecting arms.

In one embodiment of the invention, the first light source assembly is connected to the regulator, and each of the two connecting arms has a fixed end connected to the headwear device body and an adjustment end connected to the regulator, the two connecting arms being spaced apart from one another along a second direction perpendicular to the first direction, the regulator being configured to adjust a telescopic extension of the two connecting arms.

In one embodiment of the invention, the regulator comprises a first positioning structure, and each of the two connecting arms comprises a plurality of second positioning structures arranged along a length thereof, the first positioning structure being configured to cooperate with different ones of the second positioning structures to lock the two connecting arms at different extension positions.

In one embodiment of the invention, the head circumference adjustment assembly further comprises a support structure connected between the regulator and the headwear device body.

In one embodiment of the invention, the light therapy headwear device comprising a first light-transmitting shell coupled to the regulator of the head circumference adjustment assembly and disposed on a side of the first light source assembly facing away from the regulator of the head circumference adjustment assembly, wherein the first light-transmitting shell is configured to transmit light emitted by the first light source assembly to the back of the head of the user.

In one embodiment of the invention, the first light source assembly comprises a first light panel having an arc structure with an opening facing the accommodating space, and a plurality of first light therapy lamps connected to the first light panel.

In one embodiment of the invention, the light therapy headwear device comprising a power supply and a controller electrically connected to the first light source assembly, wherein the controller is configured to control an on/off state of each of the power supply and the first light source assembly, and a continuous illumination duration of the first light source assembly.

In one embodiment of the invention, the light therapy headwear device comprising a buffer component connected to an interior of the headwear device body via a hook-and-loop fastener, wherein the buffer component comprises a sponge and a mesh leather wrapped around the sponge and is configured to protect the forehead of the user.

In one embodiment of the invention, the light therapy headwear device comprising a second light-transmitting shell, a third light-transmitting shell, and a support assembly connected to the second light-transmitting shell and the third light-transmitting shell, wherein the support assembly comprises a plurality of support columns configured to define airflow channels between the second light-transmitting shell and/or the third light-transmitting shell and the head of the user for dissipating heat.

In one embodiment of the invention, the support assembly further comprises a plurality of arc-shaped limiting members, each limiting member being connected to respective ends of a plurality of the support columns proximate to a respective light source assembly.

According to a second aspect of the invention, a light therapy headwear device is provided. The light therapy headwear device comprising: a headwear device body comprising a front headwear device portion and a rear headwear device portion, the headwear device body having an accommodation opening at one end of the headwear device body along a first direction; a second light source assembly coupled to an interior of the front headwear device portion and configured to emit light toward a forehead region of a head of a user; a third light source assembly coupled to an interior of the rear headwear device portion and configured to emit light toward overhead and upper rear regions of the head of the user; and a head circumference adjustment assembly coupled to the headwear device body proximate to the accommodation opening and having a first light source assembly coupled thereto, the first light source assembly being configured to perform light therapy on a back of the head of the user, wherein the head circumference adjustment assembly is mechanically coupled to the first light source assembly and configured such that adjustment of the head circumference adjustment assembly causes displacement of the first light source assembly relative to the back of the head of the user to selectively adjust a spacing distance between the first light source assembly and the back of the head of the user, thereby regulating thermal energy transfer and light therapy intensity from the first light source assembly to the back of the head of the user.

In one embodiment of the invention, the light therapy headwear device comprising a second light-transmitting shell arranged on a side of the second light source assembly facing away from the front headwear device portion, and a third light-transmitting shell arranged on a side of the third light source assembly facing away from the rear headwear device portion.

In one embodiment of the invention, the light therapy headwear device comprising a support assembly connected to the second light-transmitting shell and/or the third light-transmitting shell, forming a first support space on the side of the second light-transmitting shell facing away from the second light source assembly, and/or forming a second support space on the side of the third light-transmitting shell facing away from the third light source assembly.

In one embodiment of the invention, the support assembly comprises a plurality of spaced-apart support columns extending away from the second light-transmitting shell and/or the third light-transmitting shell.

In one embodiment of the invention, the support assembly further comprises a plurality of arc-shaped limiting members, each limiting member being connected to the respective ends of a plurality of the support columns, the ends being proximate to the respective light source assembly.

In one embodiment of the invention, the light therapy headwear device comprising a power supply and a controller mounted to the headwear device body, the controller configured to control an on/off state of the power supply and the first, second, and third light source assemblies, and a continuous illumination duration of the first, second, and third light source assemblies, wherein the power supply and controller are electrically connected to all of the first, second, and third light source assemblies.

According to a third aspect of the invention, a method of operating a light therapy headwear device is provided. The method comprising: positioning a light therapy headwear device on a head of a user, the light therapy headwear device comprising a headwear device body, a head circumference adjustment assembly connected to the headwear device body, and a first light source assembly connected to the head circumference adjustment assembly and oriented to direct light toward a back of the head of the user; adjusting the head circumference adjustment assembly to fit the light therapy headwear device to a head circumference of the user, wherein adjusting the head circumference adjustment assembly causes displacement of the first light source assembly relative to the back of the head of the user as a direct result of the adjustment, thereby adjusting a spacing distance between the first light source assembly and the back of the head of the user to regulate thermal energy transfer and light therapy intensity to the back of the head of the user; and operating the first light source assembly to emit therapeutic light toward the back of the head of the user.

In one embodiment of the invention, adjusting the head circumference adjustment assembly comprises operating a regulator to telescopically extend or retract two connecting arms that extend laterally from the headwear device body, thereby moving the first light source assembly toward or away from the back of the head of the user.

In one embodiment of the invention, further comprising: operating a second light source assembly disposed inside a front headwear device portion of the headwear device body to direct therapeutic light toward a forehead region of the head of the user; and operating a third light source assembly disposed inside a rear headwear device portion of the headwear device body to direct therapeutic light toward overhead and upper rear regions of the head of the user, wherein the first, second, and third light source assemblies together irradiate the back, the forehead region, and the overhead and upper rear regions of the head of the user.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first’, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “helmet body” or “wearable housing” or “headwear device” refers to a structure configured to be worn on the head or another body part, designed to house and retain functional components such as light sources, sensors, and control electronics. The housing may be rigid, semi-rigid, or flexible, and may be formed from materials such as polycarbonate, EVA foam, or silicone rubber.

In the context of the specification, the term “LED module” or “light therapy lamp” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200 nm, and 1400 nm. As used herein, the term “light therapy” refers to the use of one or more light sources of any type that emit light with a wavelength between about 400 nm and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

In the context of this specification, terms like “light”, “radiation”, “irradiation”, “emission”, and “illumination”, etc., refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, Super luminous diodes (“SLD”), or Organic light-emitting diodes (OLED).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of the specification, the term “user” or “subject” is intended to broadly cover humans, animals, or other recipients of the treatment, unless otherwise specifically limited.

### BRIEF DESCRIPTION OF DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 is a side view of a light therapy headwear device, in accordance with an embodiment of the present invention.

FIG. 2 is a front view of the light therapy headwear device, in accordance with an embodiment of the present invention.

FIG. 3 is an exploded perspective view showing the first light-transmitting shell and regulator of the head circumference adjustment assembly, in accordance with an embodiment of the present invention.

FIG. 4 is an exploded perspective view of the internal structure of the headwear device body, in accordance with an embodiment of the present invention.

FIG. 5 is a perspective view of a support assembly of the light therapy headwear device, in accordance with an embodiment of the present invention.

FIG. 6 is an exploded perspective view of a rear headwear device portion and a controller of the light therapy headwear device, in accordance with an embodiment of the present invention.

### DETAILED DESCRIPTION OF THE INVENTION

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

The present invention provides a wearable light therapy headwear device configured to deliver therapeutic light treatment to multiple regions of a user's scalp and head. The device comprises a headwear device body defining an accommodating space for receiving a user's head, with an accommodation opening at the lower end thereof. The headwear device body includes a front headwear device portion and a rear headwear device portion configured to cover the forehead, sides, overhead, and rear regions of the head. A buffer component is preferably disposed on the interior surface of the front headwear device portion to provide cushioning and comfort protection for the user's forehead during use.

Throughout this specification, reference is made to a first direction Z, a second direction X, and a third direction P. The first direction Z refers generally to the direction extending from the accommodation opening at the lower end of a headwear device body to the upper end of the headwear device body, which corresponds generally to the vertical direction when the device is worn by a user in a normal upright position. The second direction X refers to the lateral (side-to-side) direction that is perpendicular to the first direction Z. The third direction P refers to the front-to-back direction that is perpendicular to the second direction X, and intersects the first direction Z. These directional references are used herein for descriptive convenience and do not constitute limitations on the orientation or configuration of the claimed invention.

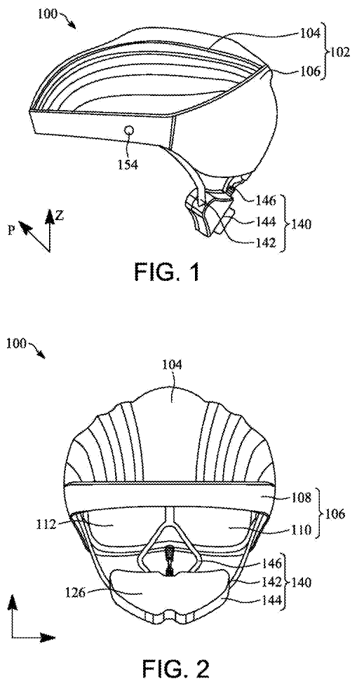

Referring to FIGS. 1 to 6, a light therapy headwear device 100 according to a preferred embodiment of the present invention is described in detail below. The light therapy headwear device 100 is a wearable therapeutic device configured to be worn on the head of a user and to deliver therapeutic light to one or more regions of the user's scalp and head. By irradiating the scalp with light of an appropriate wavelength and optical power, the light therapy headwear device 100 stimulates hair follicles, promotes local blood circulation, and supports hair growth and scalp health. The light therapy headwear device 100 is particularly suited to the treatment of hair loss conditions.

In an embodiment, the light therapy headwear device 100 comprises a headwear device body 102, a head circumference adjustment assembly 140, and a first light source assembly 116. In some embodiments, the light therapy headwear device 100 further comprises a first light-transmitting shell 126, a second light source assembly 122, a third light source assembly 124, a second light-transmitting shell 128, a third light-transmitting shell 132, a support assembly 134, a power supply 148, and a controller 150.

Referring to FIGS. 1, 2, and 4, the headwear device body 102 constitutes the primary structural frame of the light therapy headwear device 100 and serves to protect the head of the user and to provide structural support and mounting points for the various components of the light therapy headwear device 100. The headwear device body 102 is generally dome-shaped or helmet-shaped and is sized and proportioned to receive and enclose the upper portion of a human head.

The headwear device body 102 defines an accommodating space 112 that is configured to receive and accommodate the head of the user. The accommodating space 112 is bounded on its upper and lateral sides by the wall of the headwear device body 102. At the lower end of the headwear device body 102 along the first direction Z, the headwear device body 102 defines an accommodation opening that communicates with the accommodating space 112 and through which the user's head is inserted when the light therapy headwear device 100 is donned. The head circumference adjustment assembly 140 is connected to the headwear device body 102 at or proximate to the lower edge thereof, i.e., adjacent to the accommodation opening, along the first direction Z.

In an embodiment, the headwear device body 102 comprises a front headwear device portion 104 and a rear headwear device portion 106 that are connected with each other. The front headwear device portion 104 is a curved plate structure configured to cover the forehead and upper regions of the user's head. The rear headwear device portion 106 is connected to the side of the front headwear device portion 104 and is configured to cover the back, sides, and overhead region of the user's head.

In an embodiment, the rear headwear device portion 106 comprises a support edge 108 and a protective shell 110. The support edge 108 is a curved strip structure, and the protective shell 110 is a curved plate structure connected to the posterior side of the support edge 108. Together, the support edge 108 and the protective shell 110 form a ring-shaped structure. This ring-shaped structure is connected to the peripheral edge of the front headwear device portion 104, specifically at the bottom periphery of the front headwear device portion 104.

The headwear device body 102 is preferably constructed from High-grade frosted PC material. This material is selected to provide structural integrity, protection of the internal components, and user comfort. The headwear device body 102 is shaped to generally conform to the contour of the human head, providing a stable fit and aesthetically appropriate appearance.

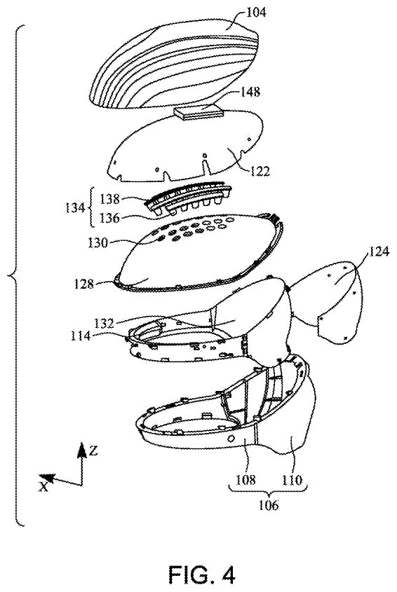

Referring to FIG. 4, the light therapy headwear device 100 includes a buffer component 114 disposed on the interior of the headwear device body 102. The buffer component 114 is preferably connected to the interior surface of the headwear device body 102 by means of a hook-and-loop fastener, allowing the buffer component 114 to be readily attached and detached for cleaning, replacement, or adjustment.

The buffer component 114 comprises a sponge and a mesh leather wrapped around the sponge. The sponge provides cushioning, while the mesh leather provides a comfortable, breathable, and hygienic surface that contacts the user's forehead. The buffer component 114 is positioned to protect the user's forehead from pressure and abrasion during use. The hook-and-loop fastener connection facilitates easy removal for cleaning and ensures that the buffer component 114 can be repositioned or replaced as needed.

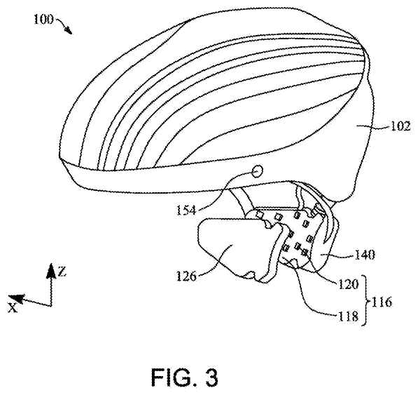

Referring to FIGS. 1, 2, and 3, the head circumference adjustment assembly 140 is connected to the headwear device body 102 at a location proximate to the lower end of the headwear device body 102 along the first direction Z. As shown in FIGS. 1 and 2, the head circumference adjustment assembly 140 hangs below the lower edge of the headwear device body 102.

The head circumference adjustment assembly 140 serves two primary functions. First, and most importantly, because the first light source assembly 116 is coupled directly to the head circumference adjustment assembly 140, adjustment of the head circumference adjustment assembly 140 adjusts the spacing distance between the first light source assembly 116 and the back of the user's head. This enables the user to directly and conveniently control the amount of heat and light intensity transmitted from the first light source assembly 116 to the posterior scalp, which is a key advancement over fixed-distance prior art configurations. Second, the head circumference adjustment assembly 140 enables the helmet to be stably worn on heads of different sizes. The headwear device body 102 has a fixed shell size that is configured to accommodate a range of head circumferences. The regulator 144 and connecting arms 142 form a rear stabilizing cradle that can be adjusted to contact the back of the user's head, thereby securing the light therapy headwear device 100 in a stable position during use.

The head circumference adjustment assembly 140 comprises a regulator 144, a pair of connecting arms 142, and a support structure 146.

The two connecting arms 142 are elongated members, each extending from the headwear device body 102 in a direction generally along the first direction Z toward the regulator 144. Each connecting arm 142 has a fixed end and an adjustment end. The fixed end of each connecting arm 142 is connected to the headwear device body 102 at a location near the accommodation opening along the first direction Z. The two connecting arms 142 are disposed in spaced-apart relation relative to each other along the second direction X, meaning that one connecting arm 142 is connected to one lateral side of the headwear device body 102, and the other connecting arm 142 is connected to the other lateral side of the headwear device body 102.

The two connecting arms 142 are adjustably engaged with the regulator 144. By operating the regulator 144, the effective length of each connecting arm 142 between its fixed end on the headwear device body 102 and its connection to the regulator 144 can be increased or decreased. As the connecting arms 142 are telescopically extended, the overall span of the assembly increases, causing the regulator 144 and the first light source assembly 116 to move away from the headwear device body 102. The actual spacing distance between the first light source assembly 116 and the user's posterior scalp is determined jointly by the telescopic extension setting of the connecting arms 142 and the circumference of the user's head. By adjusting the telescopic extension of the connecting arms 142, the user simultaneously adjusts the fit of the light therapy headwear device 100 to their head circumference and modulates the spacing distance between the first light source assembly 116 and the posterior scalp, thereby regulating thermal energy transfer and light therapy intensity from the first light source assembly 116 to the back of the head. In this way, the spacing distance between the first light source assembly 116 and the back of the head is selectively adjustable among a plurality of discrete positions.

The adjustment ends of the two connecting arms 142 are connected to the respective ends of the regulator 144 along the second direction X. The regulator 144 is thus disposed between the two connecting arms 142, and is positioned generally at the back region of the light therapy headwear device 100 when the light therapy headwear device 100 is worn. The regulator 144 is positioned at the midpoint of the light therapy headwear device 100 along the second direction X, placing it directly behind the user's head when the light therapy headwear device 100 is worn. This central positioning ensures that the first light source assembly 116 connected to the regulator 144 is centered directly opposite the midpoint of the back of the user's head so that the light emitted by the first light source assembly 116 is evenly distributed across the back of the head.

The regulator 144 is configured to adjust the length of each connecting arm 142 that extends between the headwear device body 102 and the regulator 144. By controlling the telescopic extension of the connecting arms 142, the regulator 144 controls the effective overall span of the assembly comprising the two connecting arms 142 and the regulator 144. When the overall span is extended, the distance between the regulator 144 and the headwear device body 102 is increased. This configuration accommodates users with a larger head circumference: a larger head occupies more of the accommodating space 112, and greater arm extension is required to allow the device to fit stably over the larger head. Conversely, when the overall span is retracted, the distance between the regulator 144 and the headwear device body 102 is decreased, accommodating users with a smaller head circumference who require less span to achieve a stable fit. Within a properly fitted configuration for any given user, the user may further adjust the telescopic extension of the connecting arms 142 beyond the minimum required for a stable fit. Extending the arms beyond the minimum fit increases the spacing distance between the first light source assembly 116 and the posterior scalp of the user, reducing the heat transferred to the scalp. Retracting the arms toward the minimum fit decreases this spacing distance, increasing the heat transferred to the scalp. By selecting the appropriate telescopic extension, the user can adjust the fit of the light therapy headwear device 100 and simultaneously fine-tune the spacing distance between the first light source assembly 116 and the back of the head to achieve a comfortable and therapeutically appropriate level of light and heat delivery.

In an embodiment, the regulator 144 includes a first positioning structure, and the two connecting arms 142 include a plurality of second positioning structures arranged along their respective lengths. The first positioning structure of the regulator 144 cooperates with different second positioning structures on the connecting arms 142 to lock the connection position of the regulator 144 and the connecting arms 142, effectively fixing the overall length of the head circumference adjustment assembly 140 at a selected value.

In an embodiment, the second positioning structures on the two connecting arms 142 comprise teeth arranged along the length of each connecting arm 142, and the first positioning structure of the regulator 144 comprises a gear. The gear meshes with the teeth on the connecting arms 142, and by turning the gear, the user can cause the connecting arms 142 to extend or retract, thereby adjusting the position of the regulator 144 relative to the headwear device body 102. Alternatively, the second positioning structures may comprise snap holes arranged along the length of each connecting arm 142, and the first positioning structure may comprise a snap or buckle that engages with different snap holes to lock the connecting arms 142 at different extension positions.

The regulator 144 is preferably disposed at a spacing along the first direction Z from the headwear device body 102, as best shown in FIG. 1. This spacing ensures that the first light source assembly 116, which is mounted to the regulator 144, is positioned at a suitable elevation corresponding to the back of the head when the light therapy headwear device 100 is worn.

In an embodiment, the head circumference adjustment assembly 140 further comprises a support structure 146. The support structure 146 is connected between the regulator 144 and the headwear device body 102. Specifically, one end of the support structure 146 along the first direction Z is connected to the headwear device body 102, and the other end of the support structure 146 along the first direction Z is connected to the regulator 144. The support structure 146 reinforces and stabilizes the connection between the regulator 144 and the headwear device body 102, maintaining the position of the regulator 144 relative to the headwear device body 102 to prevent the regulator 144 from shifting or tilting during use.

In an embodiment, the support structure 146 comprises two support arms, each having one end connected to the headwear device body 102 and the other end connected to the regulator 144, providing a more robust and stable connection. In other embodiments, the support structure 146 comprises a single support arm, providing a simpler structural arrangement. The support structure 146 enables the regulator 144 to remain stably positioned directly opposite the back of the user's head even while the user is adjusting the connecting arms 142, so that the first light source assembly 116 can consistently perform light therapy on the intended target area at the back of the head.

Referring to FIGS. 2 and 3, the first light source assembly 116 is connected to the regulator 144 of the head circumference adjustment assembly 140 and is correspondingly positioned at the posterior and lower side of the light therapy headwear device 100, directly opposite the back of the user's head when the light therapy headwear device 100 is worn. Because the first light source assembly 116 is coupled to the regulator 144 and moves with the regulator 144 as the connecting arms 142 are adjusted, the spacing distance between the first light source assembly 116 and the back of the user's head is simultaneously adjusted whenever the head circumference adjustment assembly 140 is adjusted to fit the user's head circumference. This enables the user to modulate the heat transmitted from the first light source assembly 116 to the posterior scalp simply by adjusting the head circumference fit of the light therapy headwear device 100, without any separate heat adjustment mechanism.

The first light source assembly 116 is configured to emit therapeutic light toward the back of the user's head, promoting blood circulation and hair follicle stimulation in the posterior scalp. In a preferred embodiment, the first light source assembly 116 is configured to emit light with a wavelength of 650 nm and an optical power density of 3-5 mW/cm2. These parameters are consistent with the therapeutic wavelengths known to promote hair growth and scalp health through photobiomodulation. The first light therapy lamps 120 may comprise laser diodes, light-emitting diodes (LEDs), or other suitable light therapy light-emitting devices operating at the therapeutic wavelength.

In an embodiment, the first light source assembly 116 comprises a first light panel 118 and a plurality of first light therapy lamps 120 connected to the first light panel 118. The first light panel 118 is configured as an arc-shaped plate structure with an opening facing the accommodating space 112. The arc configuration of the first light panel 118 conforms generally to the curvature of the back of the human head, allowing the plurality of first light therapy lamps 120 to be distributed in an arc pattern. This arc-shaped distribution ensures that each of the plurality of first light therapy lamps 120 is oriented to irradiate a respective different position on the back of the head, so that the light therapy light is evenly distributed across the entire back of the head.

In an embodiment, the opening of the first light panel 118 faces the third direction P, with the arc of the first light panel 118 curving in the third direction P, which is perpendicular to the second direction X and intersects the first direction Z.

Referring to FIGS. 2 and 3, the light therapy headwear device 100 further comprises a first light-transmitting shell 126. The first light-transmitting shell 126 is connected to the regulator 144 and is disposed on the side of the first light source assembly 116 that faces away from the regulator 144. Accordingly, the first light source assembly 116 is sandwiched between the regulator 144 and the first light-transmitting shell 126, with the first light-transmitting shell 126 facing the user's posterior scalp when the light therapy headwear device 100 is worn.

The first light-transmitting shell 126 is fabricated from a light-transmitting material that permits light emitted by the first light therapy lamps 120 of the first light source assembly 116 to pass through the first light-transmitting shell 126 and reach the user's scalp. Suitable light-transmitting materials include, but are not limited to, transparent silicone, transparent polyvinyl chloride (PVC), transparent polycarbonate, transparent acrylic, and other optically clear or translucent polymers. The material is selected to have high transmittance at the 650 nm wavelength of the therapeutic light.

In an embodiment, the first light-transmitting shell 126, the first light panel 118, and the housing of the regulator 144 are each formed as arc-shaped structures with their openings facing the third direction P, and are arranged in sequence along the third direction P. The arc-shaped geometry of the first light-transmitting shell 126 conforms to the curvature of the posterior scalp, enabling the first light-transmitting shell 126 to be maintained at a substantially uniform distance from the scalp surface across its extent.

The first light-transmitting shell 126 serves multiple important functions. It acts as a protective cover for the first light source assembly 116, shielding the first light therapy lamps 120 from mechanical damage and from contact with the user's scalp. During light therapy, the first light-transmitting shell 126 is interposed between the first light source assembly 116 and the posterior scalp of the user, physically separating the light-emitting elements from the skin. This separation reduces the direct conduction of heat from the first light therapy lamps 120 to the scalp, thereby helping to prevent thermal injury to the back of the head. The provision of the first light-transmitting shell 126 is thus an important complementary heat-management measure that works in conjunction with the adjustable distance mechanism of the head circumference adjustment assembly 140.

Referring to FIG. 4, the light therapy headwear device 100 further comprises a second light source assembly 122 and a third light source assembly 124, which, together with the first light source assembly 116 provide comprehensive light therapy coverage of the entire scalp.

The second light source assembly 122 is connected to the front headwear device portion 104 and is disposed on the interior of the front headwear device portion 104. Accordingly, when the user wears the light therapy headwear device 100, the second light source assembly 122 is located between the user's forehead region and the front headwear device portion 104. The second light source assembly 122 is configured to emit therapeutic light in the direction away from the front headwear device portion 104, which corresponds to the direction toward the user's head. The second light source assembly 122 is thus configured to irradiate the forehead region of the user's scalp. In a preferred embodiment, the second light source assembly 122 comprises a second light panel and a plurality of second light therapy lamps connected to the second light panel, enabling uniform therapeutic light delivery across the forehead region of the user's scalp. The second light source assembly 122 preferably emits light with a wavelength of 650 nm and an optical power density of 3-5 mW/cm2.

The third light source assembly 124 is connected to the rear headwear device portion 106 and is disposed on the interior of the rear headwear device portion 106. Accordingly, when the user wears the light therapy headwear device 100, the third light source assembly 124 is located between the user's head and the rear headwear device portion 106. The third light source assembly 124 is configured to emit therapeutic light in the direction away from the rear headwear device portion 106, which corresponds to the direction toward the user's head. The third light source assembly 124 is thus configured to irradiate the overhead and upper rear regions of the user's scalp. In a preferred embodiment, the third light source assembly 124 comprises a third light panel and a plurality of third light therapy lamps connected to the third light panel. The third light source assembly 124 preferably emits light with a wavelength of 650 nm and an optical power density of 3-5 mW/cm2.

Through the combined operation of the first light source assembly 116, the second light source assembly 122, and the third light source assembly 124, the light therapy headwear device 100 provides therapeutic light simultaneously and comprehensively to the back of the head, the forehead region, and the overhead and upper rear regions, substantially improving the light therapy effect.

Referring to FIG. 4, the light therapy headwear device 100 further comprises a second light-transmitting shell 128 and a third light-transmitting shell 132.

The second light-transmitting shell 128 is disposed on the side of the second light source assembly 122 that faces away from the front headwear device portion 104, i.e., on the side of the second light source assembly 122 facing the user's head. The second light-transmitting shell 128 is located within the accommodating space 112. When the user wears the light therapy headwear device 100, the second light-transmitting shell 128 is positioned between the second light source assembly 122 and the user's forehead region. The second light-transmitting shell 128 is composed of a light-transmitting material, such as, but not limited to, transparent silicone or transparent PVC, allowing the light therapy light emitted by the second light source assembly 122 to pass through the second light-transmitting shell 128 and reach the user's forehead and scalp, while simultaneously reducing the direct conduction of heat from the second light source assembly 122 to the scalp.

The third light-transmitting shell 132 is disposed on the side of the third light source assembly 124 that faces away from the rear headwear device portion 106, i.e., on the side of the third light source assembly 124 facing the user's head. The third light-transmitting shell 132 is thus located within the accommodating space 112. When the user wears the light therapy headwear device 100, the third light-transmitting shell 132 is positioned between the third light source assembly 124 and the user's overhead and upper rear scalp region. Like the second light-transmitting shell 128, the third light-transmitting shell 132 is composed of a light-transmitting material, such as, but not limited to, transparent silicone or transparent PVC, transmitting the light therapy light emitted by the third light source assembly 124 to the user's overhead and upper rear scalp while reducing direct heat conduction.

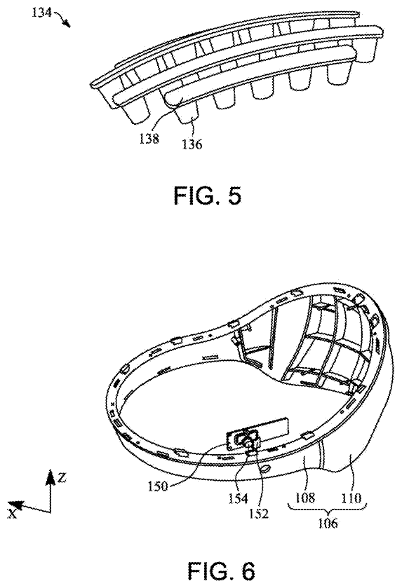

Referring to FIGS. 4 and 5, the light therapy headwear device 100 further comprises a support assembly 134. The support assembly 134 is connected to one or both of the second light-transmitting shell 128 and the third light-transmitting shell 132, and is configured to create a first support space between the second light-transmitting shell 128 and the user's scalp, and a second support space between the third light-transmitting shell 132 and the user's scalp, facilitating heat dissipation from the interior of the light therapy headwear device 100.

At least a portion of the support assembly 134 is disposed on the side of the second light-transmitting shell 128 that faces away from the second light source assembly 122, that is, on the head-facing side of the second light-transmitting shell 128. This portion of the support assembly 134 is located within the accommodating space 112, and when the user wears the light therapy headwear device 100, it is positioned between the second light-transmitting shell 128 and the user's forehead region. The support assembly 134 thus creates a first support space on the head-facing side of the second light-transmitting shell 128, between the second light-transmitting shell 128 and the user's scalp. This first support space is part of the accommodating space 112 and allows air to circulate between the scalp and the second light-transmitting shell 128, enabling heat generated by the second light source assembly 122 to be carried away from the scalp by convection and to flow out through gaps between the support assembly 134 elements to the exterior of the light therapy headwear device 100.

Similarly, at least a portion of the support assembly 134 is disposed on the side of the third light-transmitting shell 132 that faces away from the third light source assembly 124, that is, on the head-facing side of the third light-transmitting shell 132. This portion of the support assembly 134 creates a second support space on the head-facing side of the third light-transmitting shell 132, between the third light-transmitting shell 132 and the user's scalp. This second support space similarly facilitates air circulation and heat dissipation, preventing overheating of the overhead and upper rear scalp region.

The support assembly 134 comprises a plurality of support columns 136 arranged at spaced intervals, as shown in detail in FIG. 5. Each support column 136 extends from the second light-transmitting shell 128 or the third light-transmitting shell 132 (or both) in the direction toward the user's scalp. One end of each support column 136 along its length is disposed on the head-facing side of the second light-transmitting shell 128 or the third light-transmitting shell 132.

The support columns 136 are spaced apart from each other and gaps exist between adjacent support columns 136. These gaps allow the therapeutic light emitted by the second light source assembly 122 and the third light source assembly 124 to pass through the spaces between the support columns 136 and reach the scalp, thereby maintaining the light therapy coverage area. The gaps between support columns 136 also serve as ventilation channels through which warm air from the accommodating space 112 can exit the light therapy headwear device 100, significantly enhancing the cooling performance of the light therapy headwear device 100. In a preferred embodiment, when the user wears the light therapy headwear device 100, one end of each support column 136 along its length is configured to contact the user's scalp, providing a gentle massaging stimulation to the scalp and enhancing user comfort during the light therapy session.

In an embodiment, the support assembly 134 further comprises a plurality of arc-shaped limiting members 138. As shown in FIG. 5, each limiting member 138 is an arc-strip structure connected to a plurality of support columns 136 at the ends of the support columns 136 that are proximate to the respective light source assembly (i.e., on the side facing away from the first or second support space). A plurality of support columns 136 is distributed along the extension direction of each limiting member 138. The limiting members 138 serve to distribute the supporting load across multiple support columns 136, to maintain the positional arrangement of the support columns 136, and to provide a stable mounting interface for connecting the support assembly 134 to the second light-transmitting shell 128 and the third light-transmitting shell 132.

In an embodiment, as shown in FIG. 4, the second light-transmitting shell 128 is provided with a plurality of through holes 130 that extend through the thickness of the second light-transmitting shell 128. The through holes 130 are sized and positioned to accommodate the support columns 136. The limiting members 138 are arranged on the side of the through holes 130 proximate to the second light source assembly 122 (i.e., on the inner side of the second light-transmitting shell 128, so that the limiting members 138 act as a stop or retainer that prevents the support columns 136 from being completely withdrawn through the through holes 130. This arrangement maintains the support columns 136 in their operative positions extending from the head-facing side of the second light-transmitting shell 128. In embodiments where the support assembly 134 is also connected to the third light-transmitting shell 132, the third light-transmitting shell 132 is provided with through holes 130 of corresponding configuration to accommodate the support columns 136, with the limiting members 138 acting as retainers on the inner side of the third light-transmitting shell 132.

Referring to FIGS. 4 and 6, the light therapy headwear device 100 further comprises a power supply 148 and a controller 150, both of which are connected to the headwear device body 102. The power supply 148 and the controller 150 are each electrically connected to the first light source assembly 116, and in preferred embodiments are additionally electrically connected to the second light source assembly 122 and the third light source assembly 124.

The power supply 148 is a rechargeable energy storage device, such as, but not limited to, a rechargeable lithium-ion battery or other suitable battery technology. As shown in FIG. 4, the power supply 148 is disposed between the front headwear device portion 104 and the second light source assembly 122, with the front headwear device portion 104 providing structural protection to the power supply 148. The power supply 148 is capable of supplying electrical power to the first light source assembly 116, the second light source assembly 122, and the third light source assembly 124 during light therapy sessions. The provision of an integrated rechargeable power supply 148 allows the light therapy headwear device 100 to be operated in a completely wireless and portable manner, enabling the user to move freely during light therapy treatment without being tethered to a wall outlet or external power source.

The controller 150 is mounted to a lateral side of the headwear device body 102, as shown in FIG. 6, and is electrically connected to the power supply 148 and to all of the first light source assembly 116, the second light source assembly 122, and the third light source assembly 124. The controller 150 is configured to control the on/off states of the power supply 148 and the first light source assembly 116, the second light source assembly 122, and the third light source assembly 124. The controller 150 is also configured to control the continuous on-time (illumination duration) of the light source assemblies (116, 122, 124), enabling automated shut-off after a preset treatment duration to prevent excessive or prolonged light therapy exposure.

In an embodiment, the controller 150 includes a switch 154. The switch 154 is configured to control the on/off state of the internal circuit of the controller 150, which in turn controls the connection or disconnection between the power supply 148 and the first light source assembly 116, the second light source assembly 122, and the third light source assembly 124. As shown in FIG. 6, in certain embodiments, the controller 150 is connected to the lateral side of the headwear device body 102 along the second direction X, enabling the user to conveniently press the switch 154 to turn the controller 150 on or off. In certain embodiments, a power indicator light is also provided on the headwear device body 102, electrically connected to the switch 154. The power indicator light illuminates when the circuit is powered on and extinguishes when the circuit is powered off.

In an embodiment, the controller 150 further includes a charging interface 152 electrically connected to the power supply 148. The charging interface 152 enables the user to recharge the power supply 148 using a compatible charger. The charging interface 152 may be a Type-C interface, a Micro-USB interface, or any other suitable charging connector. The charging interface 152 is preferably positioned at a location on the headwear device body 102 that is accessible and convenient for the user.

In an embodiment, the light therapy headwear device 100 comprises a safety interlock configured to prevent simultaneous operation of the first light source assembly 116, the second light source assembly 122, and the third light source assembly 124 while the power supply 148 is being charged via the charging interface 152. When a charging current is detected at the charging interface 152, the controller 150 is configured to disable the light source assemblies, thereby preventing the device from being operated in a charging state.

In an embodiment, the charging interface 152 is equipped with a power indicator light. The power indicator light is electrically connected to the power supply 148 and the controller 150. When the charge level of the power supply 148 is at or above a preset threshold value, the controller 150 controls the power indicator light to remain continuously illuminated, indicating to the user that the battery is adequately charged. When the charge level of the power supply 148 falls below the preset threshold value, the controller 150 controls the power indicator light to flash, alerting the user that the battery needs to be recharged.

In an embodiment, the controller 150 further comprises a buzzer electrically connected to the switch 154. The buzzer emits an audible sound when any one of the first light source assembly 116, the second light source assembly 122, or the third light source assembly 124 is turned on or off by the switch 154, providing an audible operational status indication to the user that is particularly useful when the user cannot easily see the visual indicator.

In an alternative embodiment, the controller 150 and the power supply 148 are housed in an external controller unit that is separate from the headwear device body 102. The external controller unit is connected to the headwear device body 102 via a cable, such as a USB cable. The external controller unit may be a handheld rectangular device configured for convenient operation by the user.

The external controller unit houses the power supply 148, such as a rechargeable lithium-ion battery, and the controller circuitry. The external controller unit includes a switch for turning the device on and off, a charging interface such as a Type-C port for recharging the power supply 148, and a connection port such as a USB port for connecting to the headwear device body 102 via the cable.

In some embodiments, the external controller unit includes a display, such as a dot matrix digital display, for showing treatment information, such as a countdown timer indicating the remaining treatment time. The external controller unit may also include a buzzer that emits a sound when the device is turned on or off.

In some embodiments, the external controller unit includes a power indicator, such as a logo or indicator light, that remains steadily illuminated when the power supply 148 is adequately charged and flashes when the power supply 148 is low or charging.

It will be appreciated that the controller 150 and power supply 148 may be configured as either an integrated unit mounted to the headwear device body 102 or as a separate external unit connected to the headwear device body 102 via a cable, and both configurations are within the scope of the present invention.

In an embodiment, the light therapy headwear device 100 further comprises a wear-detection sensor electrically connected to the controller 150 and configured to detect whether the headwear device 100 is being worn on the head of the user. The controller 150 is configured to permit activation of the first light source assembly 116, the second light source assembly 122, and the third light source assembly 124 only upon receiving a wear-detection signal from the wear-detection sensor indicating that the headwear device 100 is positioned on the head of the user. In the absence of such a signal, the controller 150 is configured to prevent illumination of the light source assemblies regardless of the state of the switch 154. This arrangement prevents inadvertent activation of the light source assemblies when the device is not being worn, thereby enhancing user safety.

The operation of the light therapy headwear device 100 is as follows. The user first positions the light therapy headwear device 100 on the head by inserting the head through the accommodation opening at the lower end of the headwear device body 102, so that the head is received within the accommodating space 112 with the first light source assembly 116 oriented toward the back of the head. The user then adjusts the head circumference adjustment assembly 140 by operating the regulator 144 to telescopically extend or retract the two connecting arms 142. This adjustment simultaneously fits the light therapy headwear device 100 to the user's head circumference and adjusts the spacing distance between the first light source assembly 116 and the back of the head to a desired level. Once the head circumference adjustment assembly 140 is properly adjusted so that the light therapy headwear device 100 sits stably on the head, the user activates the light source assemblies by pressing the switch 154 on the controller 150. The controller 150 initiates the timer function, and the light source assemblies emit therapeutic light toward the respective scalp regions for the predetermined treatment duration. Upon expiration of the treatment time, the controller 150 automatically deactivates the light source assemblies. The user may then remove the light therapy headwear device 100 from the head.

The present invention possesses significant industrial applicability within the medical device and consumer healthcare industries, particularly for the treatment of hair loss conditions such as alopecia and for general scalp health maintenance. By integrating a head circumference adjustment mechanism that simultaneously regulates light therapy intensity and thermal energy transfer, the device offers a practical solution to the safety and efficacy limitations of conventional fixed-distance headwear, thereby enhancing user compliance and therapeutic outcomes. The portable configuration coupled with advanced safety features, including wear-detection sensors and charging interlocks, renders the device suitable for widespread commercial distribution for unsupervised home use, while its robust construction and comprehensive scalp coverage also support utilization in clinical or professional salon settings. Consequently, the disclosed light therapy headwear device provides a commercially viable and technically superior apparatus for delivering light therapy, fulfilling a recognized market need for adjustable, safe, and effective personal care equipment.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A light therapy headwear device comprising:
a headwear device body having an accommodating space for a head of a user and an accommodation opening at one end of the headwear device body along a first direction;
a head circumference adjustment assembly coupled to the headwear device body proximate to the accommodation opening; and
a first light source assembly coupled to the head circumference adjustment assembly and configured to perform light therapy on a back of the head of the user,
wherein the head circumference adjustment assembly is mechanically coupled to the first light source assembly and configured such that adjustment of the head circumference adjustment assembly causes displacement of the first light source assembly relative to the back of the head of the user to selectively adjust a spacing distance between the first light source assembly and the back of the head of the user, thereby regulating thermal energy transfer and light therapy intensity from the first light source assembly to the back of the head of the user.

2. The light therapy headwear device of claim 1, wherein the head circumference adjustment assembly comprises a regulator and two connecting arms.

3. The light therapy headwear device of claim 2, wherein the first light source assembly is connected to the regulator, and each of the two connecting arms has a fixed end connected to the headwear device body and an adjustment end connected to the regulator, the two connecting arms being spaced apart from one another along a second direction perpendicular to the first direction, the regulator being configured to adjust a telescopic extension of the two connecting arms.

4. The light therapy headwear device of claim 3, wherein the regulator comprises a first positioning structure and each of the two connecting arms comprises a plurality of second positioning structures arranged along a length thereof, the first positioning structure being configured to cooperate with different ones of the second positioning structures to lock the two connecting arms at different extension positions.

5. The light therapy headwear device of claim 2, wherein the head circumference adjustment assembly further comprises a support structure connected between the regulator and the headwear device body.

6. The light therapy headwear device of claim 2, further comprising a first light-transmitting shell coupled to the regulator of the head circumference adjustment assembly and disposed on a side of the first light source assembly facing away from the regulator of the head circumference adjustment assembly, wherein the first light-transmitting shell is configured to transmit light emitted by the first light source assembly to the back of the head of the user.

7. The light therapy headwear device of claim 1, wherein the first light source assembly comprises a first light panel having an arc structure with an opening facing the accommodating space, and a plurality of first light therapy lamps connected to the first light panel.

8. The light therapy headwear device of claim 1, further comprising a power supply and a controller electrically connected to the first light source assembly, wherein the controller is configured to control an on/off state of each of the power supply and the first light source assembly, and a continuous illumination duration of the first light source assembly.

9. The light therapy headwear device of claim 1, further comprising a buffer component connected to an interior of the headwear device body via a hook-and-loop fastener, wherein the buffer component comprises a sponge and a mesh leather wrapped around the sponge and is configured to protect forehead of the user.

10. The light therapy headwear device of claim 1, further comprising a second light-transmitting shell, a third light-transmitting shell, and a support assembly connected to the second light-transmitting shell and the third light-transmitting shell, wherein the support assembly comprises a plurality of support columns configured to define airflow channels between the second light-transmitting shell and/or the third light-transmitting shell and the head of the user for dissipating heat.

11. The light therapy headwear device of claim 10, wherein the support assembly further comprises a plurality of arc-shaped limiting members, each limiting member being connected to respective ends of a plurality of the support columns proximate to a respective light source assembly.

12. A light therapy headwear device comprising:
a headwear device body comprising a front headwear device portion and a rear headwear device portion, the headwear device body having an accommodation opening at one end of the headwear device body along a first direction;
a second light source assembly coupled to an interior of the front headwear device portion and configured to emit light toward a forehead region of a head of a user;
a third light source assembly coupled to an interior of the rear headwear device portion and configured to emit light toward overhead and upper rear regions of the head of the user; and
a head circumference adjustment assembly coupled to the headwear device body proximate to the accommodation opening and having a first light source assembly coupled thereto, the first light source assembly being configured to perform light therapy on a back of the head of the user,
wherein the head circumference adjustment assembly is mechanically coupled to the first light source assembly and configured such that adjustment of the head circumference adjustment assembly causes displacement of the first light source assembly relative to the back of the head of the user to selectively adjust a spacing distance between the first light source assembly and the back of the head of the user, thereby regulating thermal energy transfer and light therapy intensity from the first light source assembly to the back of the head of the user.

13. The light therapy headwear device of claim 12, further comprising a second light-transmitting shell arranged on a side of the second light source assembly facing away from the front headwear device portion, and a third light-transmitting shell arranged on a side of the third light source assembly facing away from the rear headwear device portion.

14. The light therapy headwear device of claim 13, further comprising a support assembly connected to the second light-transmitting shell and the third light-transmitting shell, forming a first support space on the side of the second light-transmitting shell facing away from the second light source assembly, and/or forming a second support space on the side of the third light-transmitting shell facing away from the third light source assembly.

15. The light therapy headwear device of claim 14, wherein the support assembly comprises a plurality of spaced-apart support columns extending away from the second light-transmitting shell and/or the third light-transmitting shell.

16. The light therapy headwear device of claim 15, wherein the support assembly further comprises a plurality of arc-shaped limiting members, each limiting member being connected to the respective ends of a plurality of the support columns, the ends being proximate to the respective light source assembly.

17. The light therapy headwear device of claim 12, further comprising a power supply and a controller mounted to the headwear device body, the controller configured to control an on/off state of the power supply and the first, second, and third light source assemblies, and a continuous illumination duration of the first, second, and third light source assemblies, wherein the power supply and controller are electrically connected to all of the first, second, and third light source assemblies.

18. A method of operating a light therapy headwear device, the method comprising:
positioning a light therapy headwear device on a head of a user, the light therapy headwear device comprising a headwear device body, a head circumference adjustment assembly connected to the headwear device body, and a first light source assembly connected to the head circumference adjustment assembly and oriented to direct light toward a back of the head of the user;
adjusting the head circumference adjustment assembly to fit the light therapy headwear device to a head circumference of the user, wherein adjusting the head circumference adjustment assembly causes displacement of the first light source assembly relative to the back of the head of the user as a direct result of the adjustment, thereby adjusting a spacing distance between the first light source assembly and the back of the head of the user to regulate thermal energy transfer and light therapy intensity to the back of the head of the user; and
operating the first light source assembly to emit therapeutic light toward the back of the head of the user.

19. The method of claim 18, wherein adjusting the head circumference adjustment assembly comprises operating a regulator to telescopically extend or retract two connecting arms that extend laterally from the headwear device body, thereby moving the first light source assembly toward or away from the back of the head of the user.

20. The method of claim 18, further comprising:
operating a second light source assembly disposed inside a front headwear device portion of the headwear device body to direct therapeutic light toward a forehead region of the head of the user; and
operating a third light source assembly disposed inside a rear headwear device portion of the headwear device body to direct therapeutic light toward overhead and upper rear regions of the head of the user,
wherein the first, second, and third light source assemblies together irradiate the back, the forehead region, and the overhead and upper rear regions of the head of the user.
